(19) United States
(12) Patent Application Publication
(10) Pub. No.: US 2012/0321577 A1
(43) Pub. Date: Dec. 20, 2012

(54) Skin-whitening composition containing tyrosinase inhibitor

(76) Inventor: Ming-Chen Lee, Taipei City (TW)

(21) Appl. No.: 13/134,773

(22) Filed: Jun. 15, 2011

(51) Int. Cl. A61K 8/97; A61Q 19/02

(52) U.S. Cl. 424/62

(57) ABSTRACT

A skin-whitening composition containing a tyrosinase inhibitor is disclosed. The skin-whitening composition includes: the tyrosinase inhibitor (CitrusC) of 0.1-2.5 wt % based on a weight of the skin-whitening composition; ascorbic acid 2-glucoside of 0.1-2.5 wt % based on the weight of the skin-whitening composition; and tranexamic acid of 0.1-2.5 wt % based on the weight of the skin-whitening composition; wherein the ingredients of the composition work synergistically on whitening skin.

### BACKGROUND OF THE INVENTION

1. Technical Field

The present invention relates to compositions for skincare, and more particularly, to a skin-whitening composition containing a tyrosinase inhibitor, being able to inhibit tyrosinase from spontaneous polymerization, thereby preventing synthesis of melanin in skin.

2. Description of Related Art

Not limited to sunny summer days, it's almost every day in a year for Asian women to fight sunshine and pursue skin whitening. As revealed in an A. C. Nielsen survey, Taiwan is second only to Korea all over the world as a place where women are fond of white skin.

Referring to FIG. 1, synthesis of melanin in melanosome relies on many enzymes for catalysis, among which the most important is tyrosinase. Tyrosinase first hydroxylates tyrosine into L-3,4-dihydroxyphenylalanine (L-dopa) and DOPA then is rapidly converted into dopaquinone, which is an intermediate exist as watershed along the course of melanin's formation. With the presence of cysteine or glutathione that provides sulfhydryl groups, dopaquinone can be converted into cysteinyldopa, and then oxidized and polymerized into soluble, yellow-brown pheomelanin. On the other hand, without the provision of sulfhydryl groups, dopaquinone can spontaneously get cyclized and become dopachrome as an orange intermediate. Dopachrome spontaneously losing its carboxyl groups generate 5,6-dihydroxyindole (DHI), which can be rapidly oxidized and polymerized into insoluble, high-molecular-weight, dark-brown DHI-melanin. Differently, when there is dopachrome tautomerase, dopachrome does not lose its carboxyl groups, and is converted into DHI-2-carboxylic acid (DHICA), which is then oxidized and polymerized into light-brown, slightly soluble, middle-sized DHICA-melanin. DHI-melanin and DHICA-melanin are both members of eumelanins, and melanin includes eumelanins and pheomelanins.

As new whitening agents have been continually found and applied, Oriental cosmetics prefer those extracted from various plants while Western cosmetics often choose those obtained biotechnologically. Nevertheless, there are some whitening agents welcome by both markets, such as levorotatory Vitamin C, Vitamin C derivatives, kojic acid, arbutin, Vitamin A, alpha hydroxy acids and some plant extract. Those pharmaceutical-grade whitening agents, like hydroquinone, azelaic acid and retinoic acid, shall be used under doctors' instructions and prescriptions. The aforementioned whitening agents are mostly competitive inhibitors for tyrosinase, and function by acting with tyrosine before tyrosinase does so, such that tyrosinase has no chance to act with tyrosine, and the formation of melanin can be eliminated.

However, these competitive inhibitors for tyrosinase can only function at low-pH conditions, and cannot inhibit melanin's formation unless they are provided in concentration high enough for them to anticipate tyrosinase in acting with tyrosine. It is problematic because higher concentration means greater irritation, which is responsible for skin allergy and disorder. In addition, these competitive inhibitors for tyrosinase are mostly light sensitive. As they are unstable when receiving light, browning (e.g. arbutin and kojic acid) or yellowing (e.g. ascorbic acid and derivatives thereof) tends to happen and causes light-exposed skin-whitening products to yellow and black. This character is unfavorable to shelf stability and adds difficulty in transportation and storage of products made therefrom.

There are also skin-whitening products each coming with two parts, namely a part for day and a part for night, which contain different ingredients. The part for day is mainly composed of ingredients for sun block, for reducing direct exposure of human skin to UV rays in daytime, thereby reducing the formation of melanin, while the part for night is aimed at repairing and whitening skin. Such dualistic products nevertheless cause inconvenience to manufacture and distribution. Moreover, the reversed use of the parts may weaken the effect of the products and, even worse, ring about adverse effects.

### SUMMARY OF THE INVENTION

The present invention provides a skin-whitening composition containing a tyrosinase inhibitor. The skin-whitening composition primarily comprises: the tyrosinase inhibitor (CitrusC) of 0.1-2.5 wt % based on a weight of the skin-whitening composition; ascorbic acid 2-glucoside of 0.1-2.5 wt % based on the weight of the skin-whitening composition; and tranexamic acid of 0.1-2.5 wt % based on the weight of the skin-whitening composition, wherein the ingredients of the composition work synergistically on whitening skin.

One feature of the present invention is that the tyrosinase inhibitor (CitrusC), ascorbic acid 2-glucoside and tranexamic acid in the composition are designed to work synergistically for whitening skin, with the best whitening effect achieved by the end of the first week of its use, and with sustained effect of reducing melanin concentration over long-term (consecutive two weeks or longer) use.

An other feature of the present invention is that the tyrosinase inhibitor (CitrusC) is employed as an effective whitening ingredient of the disclosed composition, wherein for the purpose of the present invention the tyrosinase inhibitor (CitrusC) is preferably with a pH value of 7-9, for the optimal reaction.

Still another feature of the present invention is that the tyrosinase inhibitor (CitrusC) used in the disclosed composition is a non-competitive inhibitor, and thus can be effective in inhibiting the formation of melanin even with a relatively low concentration.

Yet another feature of the present invention is that the tyrosinase inhibitor used in the disclosed composition possesses light stability, so the composition is unlikely to discolor due to its exposure in light, allowing the composition to not necessarily be protected by special light-shielding packaging.

### BRIEF DESCRIPTION OF THE DRAWINGS

The invention as well as a preferred mode of use, further objectives and advantages thereof will be best understood by reference to the following detailed description of an illustrative embodiment when read in conjunction with the accompanying drawings, wherein:

FIG. 1 illustrates the formation of melanin;

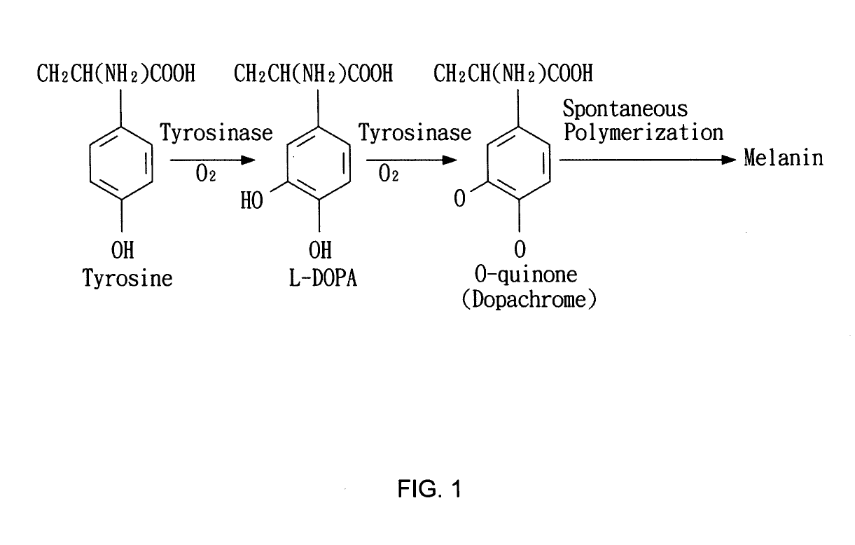

FIG. 2 is a flowchart of an experiment conducted for demonstrating the effect of the disclosed composition;

FIG. 3 is a first graph exhibiting the positive effect (%) of different groups of samples;

FIG. 4 is a second graph exhibiting the positive effect (%) of different groups of samples; and

FIG. 5 a graph exhibiting the whitening intensity (%) of different groups of samples.

### DETAILED DESCRIPTION OF THE INVENTION

The inventor of the present invention has been previously granted with U.S. Pat. No. 7,125,572 (the same invention has also been patented in Taiwan as Taiwan Patent No. 1313177). The foregoing patent involves preparing a tyrosinase inhibitor extract from lemon peels by mixing 100 g of freshly minced lemon peels with 100-1000 ml of propylene glycol, breaking cell walls of lemon peels with 1-59 seconds of sonication, sterilizing the resultant solution with microwave and removing the lemon peels with centrifugation, so the isolated supernate is the patented substance. The yield of 100 g of lemon peels extracted with propylene glycol is 70% to 90% of the volume of propylene glycol used.

The patented substance, as a tyrosinase inhibitor extracted from lemon peels, has been named by the inventor as CitrusC, which contains a protein, peptide, isoflavone and flavonoid. While it is believed that the protein is the main active component for inhibiting tyrosinase, other components are likely to provide either additive effect or anti-aging effect, so the tyrosinase inhibitor extract does not need extreme purification, therefore saving manufacturing costs.

The tyrosinase inhibitor (CitrusC) as disclosed in the inventor's U.S. Pat. No. 7,125,572 features the following characteristics:

1. Being a non-competitive inhibitor, which can effectively inhibit the formation of melanin with a relatively low concentration;

2. Presenting high inhibiting capability against tyrosinase at 25-37° C., and particularly exhibiting its maximum activity at about 25° C., while remaining 80% inhibiting capability against tyrosinase even at about 37° C. (human body temperature);

3. Having optimum reaction at pH 7-9, where the tyrosinase inhibitor extract is safe and brings no irritation or allergy to the skin when applying to skin;

4. Possessing light stability, which makes the inhibitor not to discolor under light exposure, and facilitates the inhibitor's transportation and storage without requiring special light-shielding packaging;

5. Being capable of reducing O-quinone, which is an intermediate generated during the synthetic process of melanin, thereby obstructing the synthesis of melanin and enhancing whitening effect; and

6. Being made from citrus peels, so the raw material is easy to obtain.

The patented inhibitor is extracted from citrus peels and made through biochemical reaction. The patented inhibitor contains mainly flavonoid and the peptide, wherein the peptide is the main active component for inhibiting tyrosinase and augmenting the other whitening ingredient while flavonoid providing additional effects such as anti-aging and anti-irritation. The tyrosinase inhibitor extract does not need extreme purification, therefore saving manufacturing costs.

The present invention thus uses the tyrosinase inhibitor of U.S. Pat. No. 7,125,572 (Taiwan Patent No. 1313177) as an active ingredient to develop a series of skin-whitening products and the human subject research has been conducted to prove the whitening effect of these products.

### Materials and Methodology

### I. Materials

(1) 300 samples for testing whitening effects were prepared and grouped into the following groups:

1. Group A: Creambase+Tranexamic Acid (50 samples)

2. Group B: Creambase+Tyrosinase Inhibitor (CitrusC) (50 samples)

3. Group C: Creambase+Ascorbic Acid 2-Glucoside (AA2G) (60 samples)

4. Group D: Creambase+Tranexamic Acid+Ascorbic Acid 2-Glucoside (AA2G)+tyrosinase inhibitor (CitrusC) (80 samples)

5. Group E: NIVEA Soft Moisturizing Creme (Beiersdorf AG) (30 samples), with a market price about USD. 0.033/ml

6. Group F: SHISEIDO Bio-Performance Advanced Super Revitalizer (Shiseido Co., Ltd.) (30 samples), with a market price about USD. 1.833/ml

(2) Ingredients and Formulas:

The ingredients of samples for Groups A, B, C and D are listed in Table 1, and the ingredients of samples for Groups E and F are listed in Tables 2 and 3, respectively.

(3) For preventing the subjects and testers from being affected by their own psychological expectation, the 300 samples were randomly allotted with serial numbers from No. 1 to No. 300.

TABLE 1
Ingredients of Samples for Groups A, B, C and D

| | | Group | | | |
|---|---|---|---|---|---|
| Ingredient | Function | A (%) | B (%) | C (%) | D (%) |
| Steareth-2 | Emulsifier | 4 | 4 | 4 | 4 |
| Steareth-21 | Emulsifier | 2 | 2 | 2 | 2 |
| Hydrogenated | Base | 5 | 5 | 5 | 5 |
| Polyisobutene | | | | | |
| Cetyl Alcohol | Emulsifying | 2.5 | 2.5 | 2.5 | 2.5 |
| | auxiliary | | | | |
| Stearic Acid | Base | 2.5 | 2.5 | 2.5 | 2.5 |
| Dimethicone | Facilitating | 1.5 | 1.5 | 1.5 | 1.5 |
| | permeation of | | | | |
| | active | | | | |
| | ingredients, | | | | |
| | emollient | | | | |
| BHT | Anti-oxidant | 0.01 | 0.01 | 0.01 | 0.01 |
| Cyclo- | Facilitating | 2 | 2 | 2 | 2 |
| pentasiloxane | permeation of | | | | |
| | active | | | | |
| | ingredients, | | | | |
| | emollient | | | | |
| Cyclo- | Facilitating | 1 | 1 | 1 | 1 |
| pentasiloxane | permeation of | | | | |
| & | active | | | | |
| Dimethiconol | ingredients, | | | | |
| | emollient | | | | |
| Xanthan Gum | Thickener, | 0.1 | 0.1 | 0.1 | 0.1 |
| | plant-derived | | | | |
| | colloidal | | | | |
| | polysaccharide | | | | |
| EDTA | Metal chelating | 0.03 | 0.03 | 0.03 | 0.03 |
| | agent | | | | |
| Allantoin | Skin cell | 0.2 | 0.2 | 0.2 | 0.2 |
| | proliferant | | | | |
| Water & | Water-dispersible | 1 | 1 | 1 | 1 |
| Titanium | tio2 protector | | | | |
| Dioxide & | | | | | |
| Alumina & | | | | | |
| Sodium | | | | | |
| Polyacrylate & | | | | | |
| Germaben II | | | | | |
| Dipotassium | Relaxing skin, | 0.2 | 0.2 | 0.2 | 0.2 |
| Glycyrrhizinate | anti-allergy | | | | |
| CitrusC | Low-molecular- | — | 0.1-2.5 | — | 0.1-2.5 |
| | weight peptide | | | | |
| | Tyrosinase | | | | |
| | inhibitor | | | | |
| Ascorbic Acid | Anti-oxidant | — | — | 0.1-2.5 | 0.1-2.5 |
| 2-Glucoside | | | | | |
| Tranexamic | Anti-allergy, | 0.1-2.5 | — | — | 0.1-2.5 |
| Acid | anti-irritation | | | | |
| YEAST Extract | Fermented fluid | 0.35 | 0.35 | 0.35 | 0.35 |
| | of lactic acid | | | | |
| | bacteria | | | | |
| Propylene | Water-soluble | 1 | 1 | 1 | 1 |
| glycol & | plant-derived | | | | |
| glycosphingo- | ceramide | | | | |
| lipids | | | | | |
| Poly- | Zwitterion, | 5 | 5 | 5 | 5 |
| quatermium-39 | acting as a | | | | |
| | substitute of | | | | |
| | hyaluronic acid | | | | |
| Methylparaben | Preservative | 0.1 | 0.1 | 0.1 | 0.1 |
| Dimethylol | Liquid | 0.35 | 0.35 | 0.35 | 0.35 |
| Dimethyl | preservative, | | | | |
| | being effective | | | | |
| | in inhibiting | | | | |
| | saccharomycete | | | | |
| | and mold, | | | | |
| | approved by | | | | |
| | Japan, USA, EU | | | | |
| | and ASEAN | | | | |
| Trietha- | Neutralizer | 0.2 | 0.2 | 0.2 | 0.2 |
| nolamine | | | | | |

| | |
|---|---|
| Aqua | added to 100% |

TABLE 2
Ingredients of NIVEA Soft Moisturizing Creme for Group E

| Ingredient | Feature |
|---|---|
| Aqua | Solvent |
| Paraffinum Liquidum | Solvent, antistatic agent |
| Myristyl alcohol | Emollient, emulsifier |
| Glycerin | Solvent, moisturizer |
| Butylene Glycol | Solvent, moisturizer |
| Alcohol Denat | Solvent |
| Stearic Acid | Emulsifier |
| TEA-MYRISTATE | Surfactant, emulsifier |
| Cera microcristallina | |
| Glyceryl Stearate | Emollient, emulsifier |
| Hydrogenated | Emollient |
| coco-glycerides | |
| Dimethicone | |
| Simmondsia Chinensis | |
| Tocopheryl Acetate | Moisturizer |
| Polyglyceryl-2 caprate | Emulsifier |
| Sodium carbomer | Emulsifier |
| Phenoxyethanol | Preservative |
| Lanolin alcohol | Antistatic agent, emollient, |
| | emulsifier |
| Methylparaben | Preservative |
| Butylparaben | Preservative |
| Ethylparaben | Preservative |
| Isobutylparaben | Preservative |
| Propylparaben | Preservative |
| Parfum | Fragrance |
| Linalool | Fragrance |
| Citronellol | Fragrance |
| Alpha-isomethylionone | Fragrance |
| Butylphenyl | Fragrance |
| Methylpropional | |
| Limonene | Solvent, fragrance |
| Benzyl salicylate | Sun block, fragrance, |
| | Fragrance fixative |

TABLE 3
Ingredients of SHISEIDO Bio-Performance
Advanced Super Revitalizer for Group F

| Ingredient | Feature |
|---|---|
| Citric Acid | Peeling, toner |
| Iron Oxides | Pigment |
| Saxifraga sarmentosa | Anti-oxidant |
| Tocopherol | Anti-oxidant |
| Ascorbyl Glucoside | Anti-oxidant, |
| | whitening |
| Stearyl Glycyrrhetinate | Anti-allergy |
| Squalane | Antistatic |
| | agent, |
| | moisturizer, |
| | Anti-oxidant, |
| | emollient |
| Dimethicone Copolyol | Antistatic |
| | agent, |
| | emollient |
| BHT | Anti-oxidant |
| Ethylparaben | Preservative |
| Butylparaben | Preservative |
| Phenoxyethanol | Preservative |
| Methylparaben | Preservative |
| Octyl Methoxycinnamate | Sun block |
| Stearyl Alcohol | Emulsifier |
| Propylene glycol stearate SE | Emulsifier |
| Xanthan Gum | Thickener |
| Agar | Thickener |
| Pentaerythrityl Tetraoctanoate | Moisturizer |
| Maltitol | Moisturizer |
| Tocopheryl Acetate | Anti-oxidant |
| Sodium Acetylated Hyaluronate | Moisturizer |
| Royal Jelly | Moisturizer |
| Jojoba Oil | Moisturizer, |
| | oily |
| | moisturizer |
| Mortierella Oil | Emollient |
| Phytosteryl macadamiate | Emollient |
| Rehmannia chinensis | Emollient |
| Hydrogenated c6-14 olefin polymers | Emollient, |
| | emulsifier |
| Glyceryl Stearate | Emollient, |
| | emulsifier |
| PEG-60GLYCERYL ISOSTEARATE | Surfactant |
| PEG-60 Hydrogenated Castor Oil | Surfactant |
| PEG | Surfactant, |
| | solvent, |
| | Moisturizer, |
| | emulsifier |
| Fragrance | Fragrance |
| Water | Solvent |
| Alcohol | Solvent |
| Butylene Glycol | Solvent, |
| | moisturizer |
| Glycerin | Solvent, |
| | moisturizer |
| Potassium Hydroxide | pH adjuster |
| Sodium Citrate | pH adjuster, |
| | anti-oxidant |
| Scutellaria baicalensis | Calming, |
| | allaying |
| Behenyl Alcohol | |
| Dimethicone | |
| Arginine HCl | |
| Sodium glutamate | |
| Saccharomyces lysate | |
| Paeonia suffruticosa | |
| Rosa roxburghii | |
| Zingiber Aromaticus | |
| Ginseng | |
| Trisodium EDTA | Metal |
| | chelating agent |
| Sodium hexametaphosphate | |
| Tamarix chinensis extract | |

### II. Design of Experiment

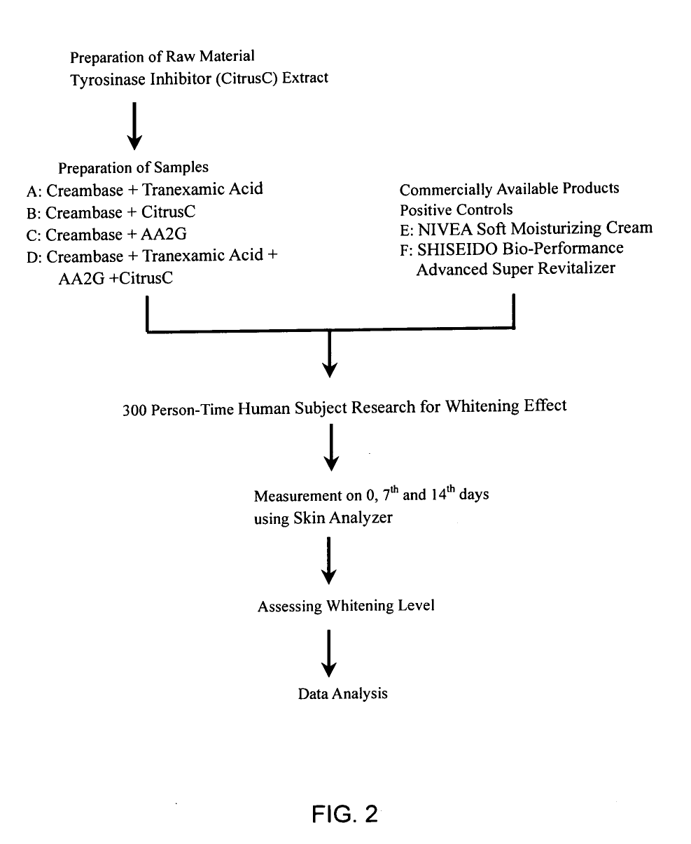

(1) The experiment was based on and modified from an experiment proposed by Sederma, the subsidiary of Merck KGaA. The flowchart of the designed experiment is shown in FIG. 2.

(2) Apparatuses
Computer: ASUS UL30A
System: Joy Silky
Device: Joy Silky Skin Analyzer provided by SILKY

### Industries Limited

(3) Subjects for Experiment

300 people (including both males and females) having ages between 18 and 30 and having healthy skin were selected from students and teachers of National Pingtung University of Science and Technology (Pingtung, Taiwan) as the subjects.

(4) Methodology

Each of the subjects had the initial skin conditions at his/her backs of both hands tested by the Skin Analyzer (DayO). The subject's left and right hands were defended as a treated area and a control area, respectively. The back of left hand was treated with a grain-size amount of the designated sample twice a day (day and night), while back of right hand was not treated. At the end of the first week (the 7^{th} day), the subjects were tested for recording the data about using the samples for seven days (D7), and at the end of the second week (the 14^{th} day), the subjects were tested for recording the data about using the samples for fourteen days (D14).

(5) Expression of Data

The data were recorded as numerals from 1 to 100. The smallest numeral means the least melanin, and the greatest numeral means the most melanin.

(6) Quantitative Assessment Formulas for Whitening Level

1. The whitening level achieved in the seven days of the first week from commencement of using the sample (T0-7):

T0−7=[(D0−D7)−(D′0−D′7)]/[(D0+D′0)/2]×100%

2. The whitening level achieved in the seven days of the second week from commencement of using the sample (T7-14):

T7−14=[(D7−D14)−(D′7−D′14)]/[(D7+D′7)/2]×100%

3. The whitening level achieved in the fourteen days of the consecutive two weeks from commencement of using the sample (T0-14):

T0−14=[(D0−D14)−(D′0−D′14)]/[(D0+D′0)/2]×100%

D0: The initial skin condition of the treated area (back of left hand) on 0 day

D7: The skin condition of the treated area (back of left hand) on the 7″ day

D14: The skin condition of the treated area (back of left hand) on the 14^{th} day

D′0: The initial skin condition of the control area (back of right hand) on 0 day

D′7: The skin condition of the control area (back of right hand) on the 7″ day

D′14: The skin condition of the control area (back of right hand) on the 14^{th} day

4. Where the results of the three formulas above are positive, it means that the sample was effective in whitening the subject's treated area. By summing up the number of samples presenting positive values in each sample group and dividing it by the effective value, the positive effect (%) can be obtained.

### III. Results and Analysis

This experiment was aimed at investigation into the whitening effects of the samples having different ingredients on human skin.

The samples to be tested were grouped into six groups. The samples of Group A were composed of creambase added with 0.1-2.5% of tranexamic acid as an anti-allergy agent. The samples of Group B and Group C were composed of creambase added with either 0.1-2.5% of tyrosinase inhibitor (CitrusC) as a whitening agent or 0.1-2.5% of ascorbic acid 2-glucoside (AA2G) as an anti-oxidant. The samples of Group D were composed of creambase added with three ingredients, namely 0.1-2.5% of tranexamic acid, 0.1-2.5% of tyrosinase inhibitor (CitrusC) and 0.1-2.5% of ascorbic acid 2-glucoside (AA2G) 3. The samples of Group E and Group F were commercially available NIVEA Soft Moisturizing Créme (sold in an open-shelf fashion) and SHISEIDO Bio-Performance Advanced Super Revitalizer (sold through cosmetics counters in department stores). Group E and Group F were positive controls in the experiment, providing objects for the comparison in whitening levels and differences among the tested samples and the commercially available products. The ingredients of the samples of Group E and Group F are listed in Table 2 and Table 3, respectively. The samples of Group E were not containing any effective whitening agent, but benzyl salicylate as UV absorber. The samples of Group F did contain ascorbic acid 2-glucoside as a whitening agent, and also contain other plant extracts for providing other effects, such as tamarix chinensis extract and extracts of ginseng, zingiber aromaticus, scutellaria baicalensis, paeonia suffruticosa and rosa roxburghii.

The 300 subjects of the experiment were selected from students and teachers of National Pingtung University of Science and Technology (Pingtung, Taiwan), including 100 males and 200 females having ages between 18 and 30. The necessary condition for the subjects to be selected was having healthy skin.

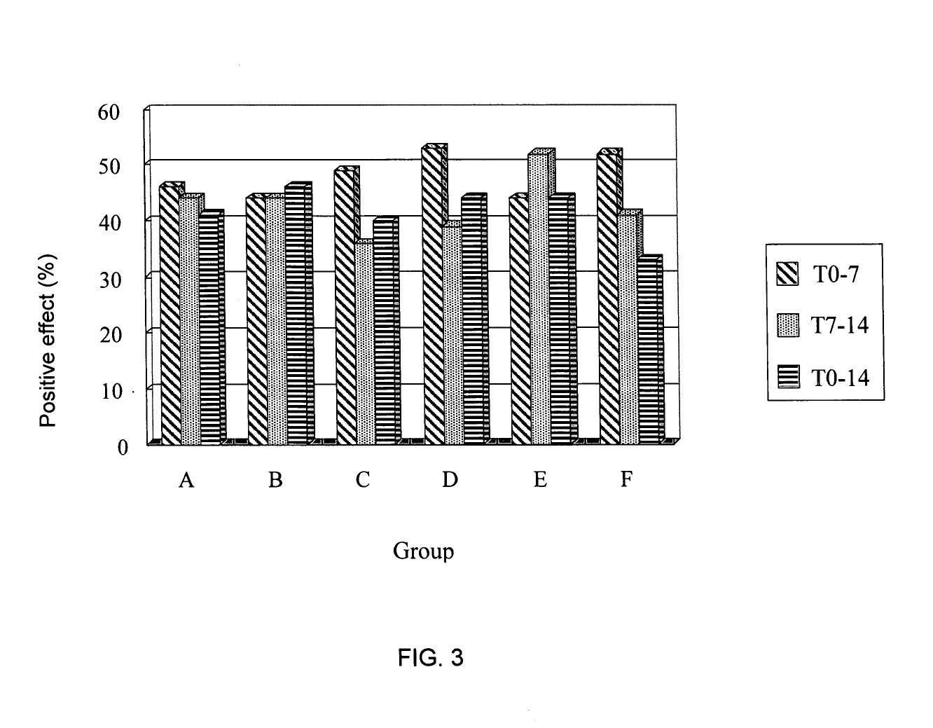

Table 4 includes data reflecting whitening effectiveness of different samples. As can be seen in FIG. 3, for the samples of Group A containing 0.1-2.5% of tranexamic acid, in the 41 subjects, 46% of them experienced whitening effected after 7 days of use, and 2% fewer in the second week as compared with that of the first week, while continuous use of 14 days only gave whitening effected to about 40% of theses subjects. Hence, the continuous use of the samples of Group A for two weeks did not contribute to an upward slope of the number of the subjects experiencing increasing whitening effect.

In the 41 subjects using the samples of Group B, 44% of them experienced whitening effect after on-week use, and the same ratio remained in the period from the 7^{th} day to the 14^{th} day, while the continuous use for two weeks gave the whitening effect to 46% of these subjects. This indicates that the samples containing 0.1-2.5% of tyrosinase inhibitor (CitrusC) when used for two successive weeks, made 5% more of the subjects have reduced melanin concentration, as compared with the sample of Group A for the same period of use. In other words, the tested tyrosinase inhibitor (CitrusC) was proven to be effective in whitening.

The samples of Group C were composed of creambase added with 0.1-2.5% of ascorbic acid 2-glucoside (AA2G). After the first week of use, the samples of this group provides 3% more positive effect as compared with those of Group A, being a minor difference. In the second week, the positive effect is 5% lower than that of the first week, and at the end of two continuous of use, only 40% of the subjects remained exhibiting the whitening effect. The results indicate that the sample containing ascorbic acid 2-glucoside (AA2G) only provided slightly better whitening effect in the first week, but lengthening the use did not increasingly add its effectiveness. It is believed that the cream containing only ascorbic acid 2-glucoside (AA2G), when used over an extended term, would have ascorbic acid 2-glucoside (AA2G) act mainly as an anti-oxidant, with the lasting of its whitening effect substantially limited.

The samples of Group D contained tranexamic acid, the tyrosinase inhibitor (CitrusC) and Ascorbic Acid 2-Glucoside (AA2G), each in an amount of 0.1-2.5%. After seven days of using the samples of Group D, the whitening effect was exhibited on 53% of the subjects, being the highest ratio among the six groups of samples. In other words, the samples containing tranexamic acid, the tyrosinase inhibitor (CitrusC) and ascorbic acid 2-glucoside (AA2G) did provide combined whitening effect. In the second 7-day period, with the disturbance of some known external factors, such as the students' frequent outdoor activities, there were still about 40% subjects remained to see the whitening effect. Considering two-week continuous use, there were still 44% subjects remained to see the reduction of melanin.

By comparing the samples of Groups A, B, C and D, it is found that after the first week of use, the samples of Group D containing tranexamic acid, the tyrosinase inhibitor (CitrusC) and ascorbic acid 2-glucoside (AA2G), each in the amount of 0.1-2.5%, provided the best whitening effect, following by the samples of Group C that contained only ascorbic acid 2-glucoside (AA2G). In the second 7-day period, the samples of Group A and Group B performed 5-8% better than those of Group C and Group D. When it comes to the continuous use for two successive weeks, Group B provided the highest positive effect, 46%, followed by the positive effect of Group D, 44%, while the Group C brought up the rear. The data reveal that the samples of Group D containing tranexamic acid, the tyrosinase inhibitor (CitrusC) and ascorbic acid 2-glucoside (AA2G), each in the amount of 0.1-2.5%, provided the synergistic whitening effect. The composition presented the best whitening effect in the first week and remained effective in reducing melanin after use for consecutive two weeks.

On the other hand, the samples of NIVEA Soft Moisturizing Creme for Group E and the samples of SHISEIDO Bio-Performance Advanced Super Revitalizer for Group F were not as effective as expected. After the first week of use, 44% subjects of Group E exhibited whitening effect, and there was an 8% increase happening in the second week. In the case of two-week continuous use, the positive effect returned to 44%. The samples of Group F provided 41˜52% positive effect in the first week and the second week, yet the positive effect thereof for two-week continuous use is the lowest one in the experiment, being 7˜13% lower than the other five groups. By studying its ingredients, it was found that there was benzyl salicylate in the samples of Group E. Benzyl salicylate is typically used in cosmetics as an UV absorber, and works by directly blocking UV rays from human skin at the starting point of the synthetic path of melanin, thus preventing formation of melanin. On the other hand, skin cells renew every 21-28 days, melanin is decomposed as cells perform metabolism. In this context, it is reasonable that the two-week continuous use of the samples of Group E provided the positive effect of 44%. Referring to the data of Group F, its two-week continuous use only provided whitening effect to one third of its subjects. While the relatively weak effect in whitening may be attributed to individual differences, living habits and/or outdoor activities, it is also noted that it contained more than 50 ingredients (Table 3), remaining it a question that whether the mutual interruption of the complex ingredients significantly affected its effect in reducing melanin. The exact reason and reactions mechanism may need further research.

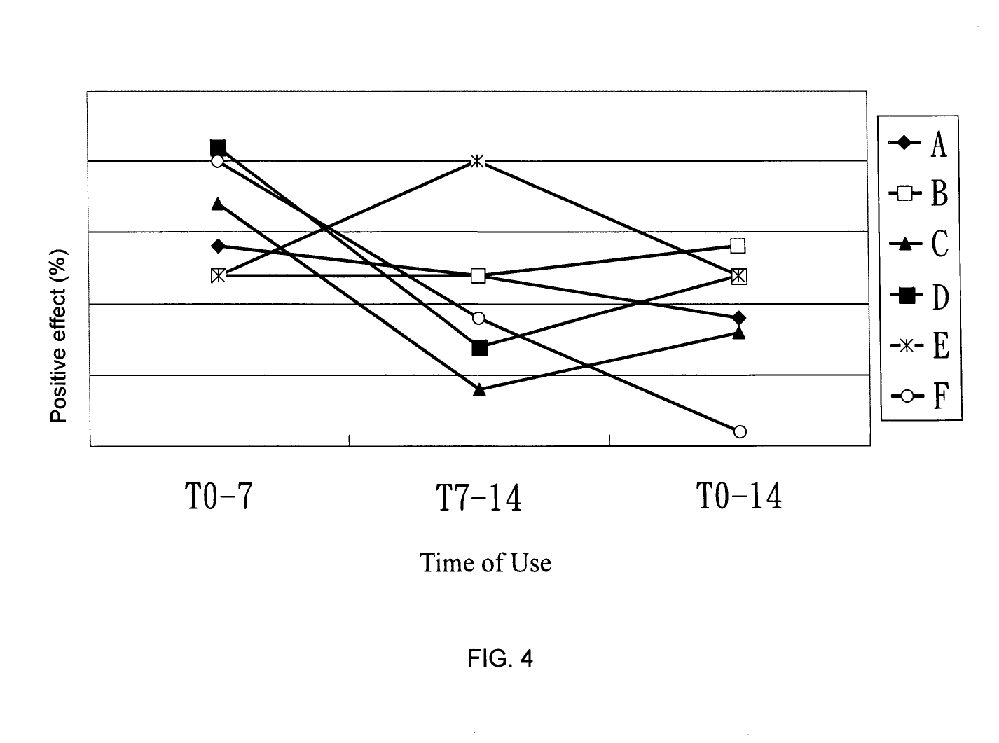

As can be seen in FIG. 4, the positive effects of Groups A, C and D all had a downward trend in the second week of use. The possible reason for this may be skin adaptability. For each of the three groups, the first week of use provided a positive effect higher than 45%, yet the positive effect in the second week decreased, implying that the treated skin got used to the ingredients and in turn the whitening effect of the samples over the lengthened period of use, so the effect of reducing melanin could not continuously increase.

TABLE 4
Experiment of Whitening Effect of Different Samples

| | | Effective | | | |
|---|---|---|---|---|---|
| | Subject | Sample | | | |
| | Size | Size | Positive Effect (%) | | |
| Group | (Person) | (Person) | T0-7* | T7-14* | T0-14* |
| (A) Creambase + | 50 | 41 | 46 | 44 | 41 |
| Tranexamic Acid | | | | | |
| (B) Creambase + | 50 | 41 | 44 | 44 | 46 |
| Tyrosinase Inhibitor | | | | | |
| (CitrusC) | | | | | |
| (C) Creambase + | 60 | 53 | 49 | 36 | 40 |
| Ascorbic Acid | | | | | |
| 2-Glucoside (AA2G) | | | | | |
| (D) Creambase + | 80 | 75 | 53 | 39 | 44 |
| Tranexamic Acid + | | | | | |
| Ascorbic Acid | | | | | |
| 2-Glucoside | | | | | |
| (AA2G) + Tyrosinase | | | | | |
| Inhibitor (CitrusC) | | | | | |
| (E) NIVEA Soft | 30 | 27 | 44 | 52 | 44 |
| Moisturizing Creme | | | | | |
| (F)SHISEIDO | 30 | 27 | 52 | 41 | 33 |
| Bio-Performance | | | | | |
| Advanced Super | | | | | |
| Revitalizer | | | | | |

*T0-7 covering the seven days of the first week from commencement of using the sample, T7-14 covering the seven days of the second week from commencement of using the sample, T0-14 covering the fourteen days of the consecutive two weeks from commencement of using the sample

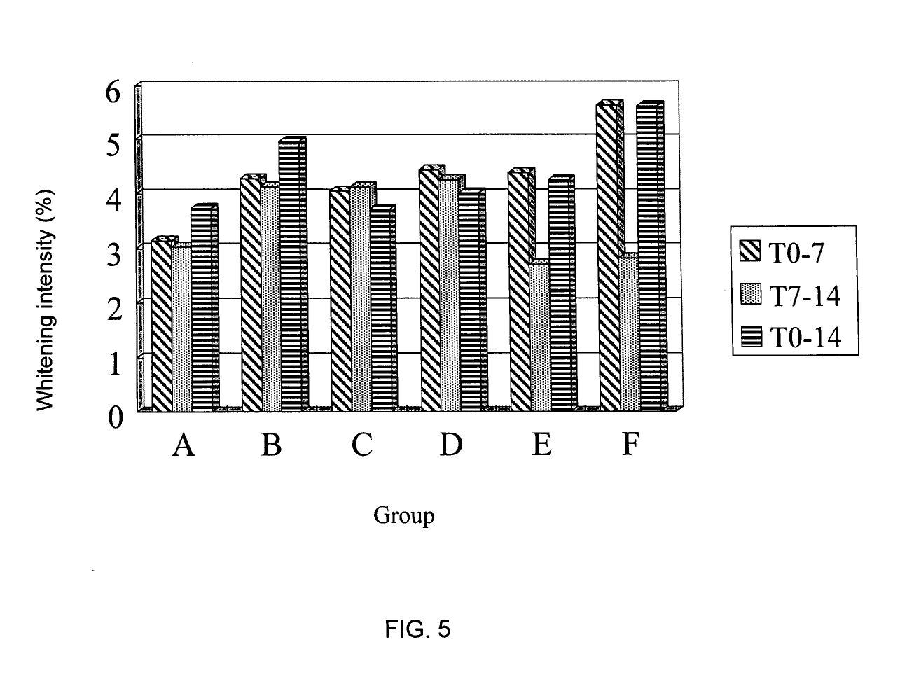

The trends of the whitening levels (indexes) of different groups can be seen clearly in Table 5 and FIG. 5. After the use of the samples of the groups for one week, the resultant whitening levels of the front 4 groups were consistent with the inventor's expatiation. Group A was the worst one. Group B provided whitening effect in virtue of the addition of tyrosinase inhibitor (CitrusC). Group C also provided meaningful effect in virtue of the addition of ascorbic acid 2-glucoside (AA2G), but was inferior to tyrosinase inhibitor (CitrusC). As proven by the data of Group D, the joint addition of tranexamic acid, the tyrosinase inhibitor (CitrusC) and ascorbic acid 2-glucoside (AA2G) gave the effect better than their separate addition. On the other hand, the both commercially available products used in the experiment as the positive controls provided superior whitening effect, particularly SHISEIDO Bio-Performance Advanced Super Revitalizer (Group F). However, the whole set of data indicate that the samples of all the groups had their whitening effect degraded in the second week of use, except those of Group B containing tyrosinase inhibitor (CitrusC), which remained effective in the second week and even presented enhanced effect. The commercially available products as the two positive controls, surprisingly had their whitening effect in the second week far behind the other groups, with the reason remaining unknown. The story, however, changed when it comes to the whitening effect of continuous use for 14 days. Therein, the positive control, Group F, gave the best effect with durable stability. The other commercially available product, Group E, also performed well. To sum up, in the experiment, the samples of Group D presented the best whitening effect with respect to time duration and stability.

TABLE 5
Whitening Effect of Different Samples for Different Time Periods

| | Whitening intensity (%) | | |
|---|---|---|---|
| Group | T0-7* | T7-14* | T0-14* |
| (A) Creambase + Tranexamic Acid | 3.14 | 3.03 | 3.73 |
| (B) Creambase + Tyrosinase Inhibitor | 4.28 | 4.69 | 4.97 |
| (CitrusC) | | | |
| (C) Creambase + Ascorbic Acid | 4.06 | 4.12 | 3.71 |
| 2-Glucoside (AA2G) | | | |
| (D) Creambase + Tranexamic Acid + | 4.44 | 4.26 | 4.02 |
| Ascorbic Acid 2-Glucoside (AA2G) + | | | |
| Tyrosinase Inhibitor (CitrusC) | | | |
| (E) NIVEA Soft Moisturizing Creme | 4.39 | 2.71 | 4.26 |
| (F)SHISEIDO Bio-Performance | 5.64 | 2.82 | 5.62 |
| Advanced Super Revitalizer | | | |

*T0-7 covering the seven days of the first week from commencement of using the sample, T7-14 covering the seven days of the second week from commencement of using the sample, T0-14 covering the fourteen days of the consecutive two weeks from commencement of using the sample

### IV. Conclusion

(1) Group D in virtue of the joint addition of tranexamic acid, the tyrosinase inhibitor (CitrusC) and ascorbic acid 2-glucoside (AA2G), each in the amount of 0.1-2.5%, provided the whitening effect enhanced by the synergistic effect among the ingredients, and thus presented the best effect in the first week of use. Even after the use of two consecutive weeks, it still performed well in reducing melanin, being comparable with the tested commercially available products. It is believed that the composition of Group D is preferable and worth commercialization.

The composition of the present invention uses the tyrosinase inhibitor (CitrusC) to inhibit action of tyrosinase directly, so as to prevent synthesis of melanin and facilitate skin whitening. Different from the conventional skin-whitening products, the disclosed composition has the tyrosinase inhibitor (CitrusC) only acting as a catalyst for chemical reaction, but not acting with and being consumed by tyrosinase. Thus, the tyrosinase inhibitor (CitrusC) can provide sufficient whitening effect at a low concentration.

Therein, ascorbic acid 2-glucoside is a derivative of Vitamin C and has been proven in lab as being effective in reducing synthesized melanin. However, it is less absorbable to human skin because of its high molecular weight. As demonstrated in the conducted human subject research, the sole addition of ascorbic acid 2-glucoside did not show good whitening effect. However, since it is relatively easy to obtain, it is used in the present invention to protect isoflavone of the tyrosinase inhibitor (CitrusC) form oxidization, thus facilitating the storage of the disclosed composition.

In addition, the tyrosinase inhibitor (CitrusC) is a natural extract, and the protein it contains may bring about allergic reaction when touching human skin. Therefore, the present invention also employs tranexamic acid, which has been highly commercialized and highly available, for the purposes of preventing allergy and irritation. Conventionally, tranexamic acid can only give whitening effect with a concentration higher than 3%. As the present invention only uses it for preventing allergy and irritation but not whitening, the amount used here is only 0.1-2.5%.

(2) The samples of Group E contained benzyl salicylate, which directly blocks UV rays from human skin at the starting point of the synthetic path of melanin, thus preventing formation of melanin. In addition, melanin is decomposed as cells perform metabolism. As a combined result, Group E remained a 44% positive effect in reducing melanin, equaling the disclosed composition.

(3) There were only one third of the subjects of Group F remaining seeing whitening effect at the end of the two-week continuous use. Although the possible reasons may extensively include individual differences, living habits and/or outdoor activities, it is also noted that it contained more than 50 ingredients. As the composition was so complicated, it is possible that the mutual interruption of the ingredients significantly affected its effect in whitening.

(4) It has been confirmed that the samples of Group D performed best in the experiment with respect to both duration and stability.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A skin-whitening composition containing a tyrosinase inhibitor, the skin-whitening composition primarily comprising:
0.1-2.5 wt % of the tyrosinase inhibitor (CitrusC), based on a weight of the skin-whitening composition;
0.1-2.5 wt % of ascorbic acid 2-glucoside, based on the weight of the skin-whitening composition; and
0.1-2.5 wt % of tranexamic acid, based on the weight of the skin-whitening composition,
wherein the ingredients of the composition work synergistically on whitening skin.

2. The skin-whitening composition of claim 1, wherein the tyrosinase inhibitor (CitrusC) comprises the following characteristics:
(1) having an highest inhibiting activity against tyrosinase at a temperature between 25° C. and 37° C.; and
(2) having an optimal pH between 7.0 and 9.0.

3. The skin-whitening composition of claim 1, wherein the tyrosinase inhibitor (CitrusC) is obtainable by a process comprising steps of:
(1) mincing lemon peels;
(2) well mixing the minced lemon peels with propylene glycol in a ratio of the minced lemon peels to the propylene glycol solution ranges from 1:1 to 1:10 into a mixture, wherein the minced lemon peels are counted by weight (grams), and the propylene glycol is counted by volume (milliliters);
(3) breaking cell walls of the minced lemon peels in the mixture;
(4) sterilizing the mixture after the step (3); and
(5) performing centrifugation to the mixture and obtaining a supernate of the mixture as the tyrosinase inhibitor (CitrusC).
